(19) United States
(12) Patent Application Publication
(10) Pub. No.: US 2009/0087440 A1
(43) Pub. Date: Apr. 2, 2009

(54) Methods for Treating Cancer

(75) Inventors: Alain Vicari, La Tour de Salvagny (FR); Christophe Caux, Bressolles (FR)

Correspondence Address: DNAX RESEARCH INC.;LEGAL DEPARTMENT, 901 CALIFORNIA AVENUE, PALO ALTO, CA, 94304, US

(73) Assignee: SCHERING CORPORATION, Kenilworth, NJ (US)

(21) Appl. No.: 12/234,361

(22) Filed: Sep. 19, 2008

Related U.S. Application Data
(63) Continuation of application No. 10/304,616, filed on Nov. 26, 2002.
(60) Provisional application No. 60/333,434, filed on Nov. 27, 2001.

(51) Int. Cl. A61K 39/395; A61P 35/00; A61K 31/7088

(52) U.S. Cl. 424/141.1; 424/130.1; 514/44

(57) ABSTRACT

Dendritic cells (DC) play a critical role in antigen-specific immune responses. Materials and Methods are provided for treating disease states, including cancer, by activating dendritic cells from the host which are rendered hypo-responsive to activation stimuli by the disease. In particular, methods are provided for treating cancer in a mammal comprising administering to said mammal an effective amount of a tumor-derived DC inhibitory factor antagonist in combination with an effective amount of a Toll-like receptor (TLR) agonist.

This application is a Continuation of U.S. patent application Ser. No. 10/304,616, filed Nov. 26, 2002, which claims benefit of U.S. Provisional Patent Application No. 60/333,434, filed Nov. 27, 2001, each of which is incorporated herein by reference.

### FIELD OF THE INVENTION

The invention relates to methods for the manipulation and activation of dendritic cells (DC) in the treatment of disease states, especially cancer.

### BACKGROUND OF THE INVENTION

Dendritic cells (DC) play a crucial role in initiating and modulating innate and adaptive immune responses (Banchereau et al., 1998, Nature 392:245-252). In the context of cancer, dendritic cells are able to sample and present tumor antigens and prime tumor-specific cytotoxic T cells (Chiodoni et al., 1999, J. Exp. Med. 190:125-133). In addition, dendritic cells can be an important source of the cytokines Interleukin-12 (IL-12), Tumor Necrosis Factor alpha (TNFα), and Interferon alpha (IFNα) which play a role in anti-tumor immune responses (Banchereau et al., 1998, Nature 392:245-252). Thus, in recent years, investigators have attempted to exploit the activity of DC in the treatment of cancer (See, e.g., Mehta-Damani et al., 1994, J. Immunology 153:996-1003; Hsu et al., 1996, Nature Medicine 2:52; Murphy et al., 1996, The Prostate 29:371; Mehta-Damani et al., 1994, J. Immunology 153:996-1003; Dallal et al., 2000, Curr. Opin. Immunol. 12: 583-588; Zeid et al., 1993, Pathology 25:338; Furihaton et al., 1992, 61:409; Tsujitani et al., 1990, Cancer 66:2012; Gianni et al., 1991, Pathol. Res. Pract. 187:496; Murphy et al., 1993, J. Inv. Dermatol. 100:3358).

To induce a proper immune response, dendritic cells must be recruited at the site of antigen expression, uptake antigens, and migrate to secondary lymphoid organs while receiving activation signals delivered by pathogens, dying cells and/or T cells. Several studies have addressed the status of DC in human tumors and have reported impaired DC functions within tumors or in cancer patients (Bell et al., 1999, J. Exp. Med. 190:1417-1426; Scarpino et al., 2000, Am. J. Pathol. 156:831-837; Lespagnard et al., 1999, Int. J. Cancer 84:309-314; Enk et al., 1997, Int. J. Cancer 73:309-316). Furthermore, the observation of activated DC in some studies was a positive prognosis factor (Enk et al., 1997, Int. J. Cancer 73:309-316). Thus, enhancing the activation of dendritic cells in tumors could be a useful method to treat cancer.

Tumors can escape the immune system by interfering with the navigation of DC or by failing to provide the necessary activation signals (Vicari et al, 2001, Seminars in Cancer Biology, in press). In particular, it is likely that tumors do not express many of the Pathogen Associated Molecular Patterns (PAMPs) (Medzhitov et al., 2000, Sem. Immunol. 12: 185-188), which trigger DC activation (Reis et al., 2001, Immunity 14: 495-498).

In recent years, the Toll-like receptor (TLR) molecules have been identified as an important class of receptors for PAMPs. Toll-like receptors (TLRs) recognize molecular patterns specific to microbial pathogens (Aderem et al., 2000, Nature 406:782-787). Ten distinct TLR molecules have been described in man. WO 98/50547, published Nov. 12, 1999, discloses TLRs 2-10. Of note, the current public nomenclature include ten distinct TLRs in man, nine of them corresponding to TLR-2 to TLR-10 of WO 98/50547 but with mismatched numbers (Kadowaki et al., 2001, J. Exp. Med. 194: 863-869).

Signaling through TLRs triggered by microbial molecules strongly activate DCs to upregulate costimulatory molecules (CD80 and CD86) (Hertz et al., 2001, J. Immunol. 166:2444-2450) and to produce proinflammatory cytokines (TNF-α, IL-6, and IL-12) (Thoma-Uszynski et al., 2001, J. Immunol. 154:3804-3810). Numerous studies have now identified a wide variety of chemically-diverse bacterial products that serve as ligands for TLR proteins, including bacterial lipo-polysaccharide (TLR-4), flagellin (TLR-5), lipoteichoic acid (TLR-2) and Poly I:C (TLR-3). More particularly, TLR-9 has been shown to be a ligand for immuno-stimulatory bacterial CpG DNA (Hemmi et al., 2000, Nature 408: 740745; Wagner, 2001, Immunity 14: 499-502).

Moreover, tumors promote the secretion of factors that inhibit DC differentiation or functions. One of the tumor-associated factors that could inhibit DC function in cancer is IL-10. It has been reported that numerous human primary tumors or metastases secrete Interleukin-10 (IL-10) (Chouaib et al., 1997, Immunol. Today 18:4993-497). This factor has been described as a strong modulator of DC function. Indeed, IL-10 can negatively regulate IL-12 production and inhibit the T-cell co-stimulatory potential of DC (DeSmedt et al., 1997, Eur. J. Immunol. 27:1229-1235; Caux et al., 1994, Int. Immunol. 6:1177-1185). The effect of antagonizing DC inhibitory signals such as IL-10 to improve DC activation and therefore the host immune response against cancer, however, is yet unknown.

The currently available methods of cancer therapy such as surgical therapy, radiotherapy, chemotherapy, and immunobiological methods have either been of limited success or have given rise to serious and undesirable side effects. In many clinically diagnosed solid tumors (in which the tumor is a localized growth), surgical removal is considered the prime means of treatment. However, many times after surgery and after some delay period, the original tumor is observed to have metastasized so that secondary sites of cancer invasion have spread throughout the body and the patient subsequently dies of the secondary cancer growth. Although chemotherapy is widely used in the treatment of cancer, it is a systemic treatment based usually on the prevention of cell proliferation. Accordingly, chemotherapy is a non-specific treatment modality affecting all proliferating cells, including normal cells, leading to undesirable and often serious side effects.

Thus, a need exists for new methods for treating diseases thought to result from aberrant immune responses, especially cancer. In particular, elucidation of the factors that facilitate the activation of tumor-infiltrating dendritic cells would allow manipulation of dendritic cells to enhance a tumor-specific immune response. Methods and therapies for the modulation of the immune response through the manipulation of dendritic cells will be useful in the treatment of these diseases.

### SUMMARY OF THE INVENTION

The present invention fulfills the foregoing need by providing materials and methods for immunotherapy for diseases such as cancer by facilitating the activation of tumor-infiltrating dendritic cells. It has now been discovered that combined administration of an IL-10 antagonist and a TLR-9 agonist is an effective cancer therapy. The invention thus provides a method of treating cancer comprising administering to an individual in need thereof an effective amount of a tumor-derived DC inhibitory factor antagonist in combination with an effective amount of a TLR agonist.

In preferred embodiments, the tumor-derived DC inhibitory factor antagonist can be an antagonist of any of the following tumor-associated factors which are known to inhibit dendritic cell function: IL-6, VEGF, CTLA-4, OX-40, TGF-β, prostaglandin, ganglioside, M-CSF and IL-10. More preferably, the tumor-derived DC inhibitory factor antagonist is an IL-10 antagonist. Most preferably, the IL-10 antagonist is either a direct antagonist of the IL-10 cytokine or an antagonist of the IL-10 receptor. In certain embodiments, the tumor-derived DC inhibitory factor antagonist is an antibody or antibody fragment, a small molecule or antisense nucleotide sequence. Most preferably, the tumor-derived DC inhibitory factor antagonist is an anti-IL-10 receptor antibody.

In certain embodiments, the TLR agonist is a small molecule, a recombinant protein, an antibody or antibody fragment, a nucleotide sequence or a protein-nucleic acid sequence. In preferred embodiments, the TLR agonist is an agonist of TLR-9. More preferably, the TLR agonist is an immunostimulatory nucleotide sequence. Still more preferably, the immunostimulatory nucleotide sequence contains a CpG motif. Most preferably, the immunostimulatory nucleotide sequence is selected from the group consisting of: CpG 2006 (Table 2 and SEQ ID NO: 1); CpG 2216 (Table 2 and SEQ ID NO: 2); AAC-30 (Table 2 and SEQ ID NO: 3); and GAC-30 (Table 2 and SEQ. ID NO.: 4). The immunostimulatory nucleotide sequence may be stabilized by structure modification such as phosphorothioate modification or may be encapsulated in cationic liposomes to improve in vivo pharmacokinetics and tumor targeting.

In certain embodiments, the tumor-derived DC inhibitory factor antagonist and/or TLR agonist are administered intravenously, intratumorally, intradermally, intramuscularly, subcutaneously, or topically.

In some embodiments, the tumor-derived DC inability factor antagonist and the TLR agonist are administered in the form of a fusion protein or are otherwise linked to each other.

The methods of the invention may further comprise administration of at least one tumor-associated antigen. The tumor antigen may be delivered in the form of a fusion protein or may be linked to the TLR agonist and/or the tumor-derived DC inhibitory factor antagonist.

In yet another aspect of the invention, an activating agent such as TNF-α, IFN-α, RANK-L or agonists of RANK, CD40-L or agonists of CD40, 41BBL or agonists of 41BB or other putative ligand/agonist of members of the TNF/CD40 receptor family is also administered.

In yet another aspect of the invention, cytokines are administered in combination, either before or concurrently, with the tumor-derived DC inhibitory factor antagonist and/or TLR agonist. In one preferred aspect, the cytokines are GM-CSF or G-CSF or FLT-3L, either used as recombinant proteins or recombinant fusion proteins or delivery vectors. Administration of these factors stimulates the generation of certain subsets of DC from precursors, thereby increasing the number of tumor infiltrating dendritic cells amenable for activation with the combination of tumor-derived DC inhibitory factor antagonist and TLR agonist.

In yet another aspect of the invention, selected chemokines are administered, either before or concurrently, with the tumor-derived DC inhibitory factor antagonist and/or TLR agonist. In one preferred aspect, the chemokines are selected from the group of CCL13, CCL16, CCL7, CCL19, CCL20, CCL21, CXCL9, CXCL10, CXCL11, CXCL12, either used as recombinant proteins or recombinant fusion proteins or delivery vectors. In a most preferred aspect, the chemokine is delivered to the tumor either directly following intra-tumor injection, or via a targeting construct such as a recombinant antibody, or via encapsulation in particular vesicles enabling a preferential delivery into tumors. Administration of chemokines can promote the recruitment of certain subsets of DC into the tumor, thereby increasing the number of tumor infiltrating dendritic cells amenable for activation with the combination of tumor-derived DC inhibitory factor antagonist and TLR agonist.

### BRIEF DESCRIPTION OF THE FIGURES

FIG. 1 shows that C26-6CK tumor-infiltrating dendritic cells are unresponsive to the combination of LPS+anti-CD40+IFNγ when compared to bone marrow-derived dendritic cells. FIG. 1A depicts the results of analysis of surface expression of MHC class II, CD40 and CD86 by FACS (gated on CD11c positive cells). FIG. 1B depicts intracellular expression of IL-12p40 by CD11c+ cells after 20 hours, including 2.5 hour incubation with Brefeldin A. FIG. 1C depicts a mixed leukocyte reaction. In FIG. 1D, IL-12 p70 was measured in culture supernatants after activation with LPS+IFNγ+anti-CD40 by a specific ELISA.

FIG. 2. CpG 1668+anti-IL-10R combination restored IL-12 and TNFα in C26-6CK tumor-infiltrating dendritic cells. TIDC from C26-6CK tumors were enriched using anti-CD11c magnetic beads and cultured overnight in the presence of GM-CSF and various combinations of LPS, IFNγ anti-CD40, anti-IL10R and CpG 1668. The levels of IL-12 p70 and TNFα were measured in culture supernatants by specific ELISA.

FIG. 3. CpG 1668+anti-IL-10R combination restored the MLR stimulatory capacity of DC infiltrating C26-6CK tumors. TIDC from C26-6CK tumors were enriched using anti-CD11c magnetic beads and cultured overnight in the presence of GM-CSF and various combinations of LPS, IFN-γ, anti-CD40, anti-IL10R and CpG 1668. Cells were then irradiated and cultured for 5 days at varying numbers in the presence of a constant number of enriched allogeneic T cells (3×105 T cells). Proliferation was measured during the last 18 hours of culture by radioactive thymidine incorporation.

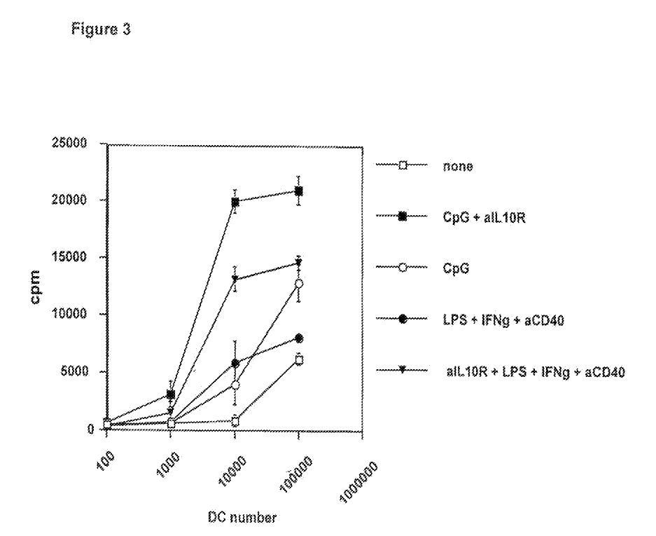

FIG. 4. Tumor-infiltrating dendritic cells from parental C26 tumors as well as from tumors of different histological origin are unresponsive to the combination of LPS+IFNγ+anti-CD40 but produce IL-12 in response to CpG 1668+anti-IL-10R. TIDC from indicated tumors were enriched using anti-CD11c magnetic beads and cultured overnight in the presence of GM-CSF, LPS+IFNγ+anti-CD40 or anti-IL10R+CpG 1668. FIG. 4 depicts intracellular expression of IL-12p40 and surface expression of CD11c in cultured cells after 20 hours, including a 2.5 hour incubation with Brefeldin A.

FIG. 5 depicts the therapeutic effect of CpG1668+anti-IL10R antibody in the C26-6CK tumor model. Groups of 7 week old female BALB/c mice were injected subcutaneously with 5×10⁴ C26-6CK cells and treated twice a week with combinations of intraperitoneal injection of 250 μg purified anti-IL10R antibody and weekly with intra-tumor injection of 10 μg CpG 1668, for three weeks starting at day 7 after tumor inoculation.

FIG. 6 depicts the therapeutic effect of CpG 1668+anti-IL10R antibody in the C26 tumor model. Groups of 7 week old female BALB/c mice were injected subcutaneously with 5×10⁴ C26 cells and treated weekly with combinations of intraperitoneal injection of 250 μg purified anti-IL10R antibody and intra-tumor injection of 5 μg CpG 1668, for three weeks starting at day 7 after tumor inoculation.

FIG. 7 depicts the therapeutic effect of CpG 1668+anti-IL10R antibody in the B1F0 melanoma tumor model. Groups of 7 week old female C57BL/6 mice were injected subcutaneously with 5×10⁴ B16F0 cells and treated weekly with combinations of intraperitoneal injection of 250 μg purified anti-IL10R antibody and intra-tumor injection of 5 μg CpG 1668, for three weeks starting at day 7 after tumor inoculation.

FIG. 8 depicts that another IL-10 antagonist, a monoclonal anti-IL10 antibody, can induce, in combination with the TLR-9 agonist CpG 1668, the production of IL-12 by DC infiltrating C26-6CK tumors. TIDC from C26-6CK tumors were enriched using anti-CD11c magnetic beads and cultured overnight in the presence of GM-CSF or anti-IL10R+CpG 1668 or anti-IL10+CpG 1668. FIG. 8 depicts intracellular expression of IL-12p40 and surface expression of CD11c in cultured cells after 20 hours, including a 2.5 hour incubation with Brefeldin A.

FIG. 9 depicts that another tumor-derived DC inhibitory factor, PGE₂, can be antagonized in order to allow for DC activation. Bone marrow-derived DC were cultured in the presence or absence of a tumor supernatant that contained (indomethacin-treated) PGE2. The different DC were than examined for the expression of maturation markers and IL-12 production, following activation with combinations of LPS, IFNγ and anti-CD40 antibody in the presence or absence of anti-IL10R antibody.

FIG. 10 depicts the therapeutic effect of CpG 1668+indomethacin in the C26-6CK colon carcinoma tumor model. Groups of 8 week old female BALB/c mice were injected subcutaneously with 5×10⁴ C26-6CK cells and treated weekly with combinations of intra-tumor injection of 5 μg CpG 1668, for three weeks starting at day 7 after tumor inoculation, and/or indomethacin, 5 μg/ml in drinking water from Day 5 to Day 28.

### DETAILED DESCRIPTION OF THE INVENTION

All references cited herein are incorporated in their entirety by reference.

The present invention is based, in part, on the surprising discovery that the combined administration of a tumor-derived DC inhibitory factor antagonist and a TLR agonist has strong therapeutic activity in several in vivo models of tumor development including C26-6CK, C26 and B16F0. It has now been discovered that combined administration of an IL-10 antagonist and a TLR-9 agonist enables tumor-infiltrating dendritic cells, otherwise refractory to activation, to produce IL-12 and TNFα and to induce improved tumor antigen-specific immune responses. Furthermore, it has now been discovered that administration of an IL-10 antagonist and a TLR-9 agonist to tumor-bearing animals could induce the rejection of the tumors.

A number of reports have addressed the activation status of DC within tumors. In one such report, mouse C26 colon carcinoma tumors transduced to express GM-CSF and CD40L were heavily infiltrated by DC with a mature phenotype, and a proportion of tumors regressed after initial growth (Chiodoni et al, 1999, J. Exp. Med. 190:125-133). The same C26 cells engineered to express 6Ckine were infiltrated by immature DC (Vicari et al., 2000, J. Immunol. 165:1992-2000). Since the activation and subsequent maturation of DC are crucial events for the initiation of the immune response, it was thought that activation of C26-6CK tumor-infiltrating dendritic cells could lead to tumor rejection. Unexpectedly, it was found that those tumor-infiltrating DC did not respond to stimulation through CD40 via an anti-CD40 agonist antibody, using as read-out the up-regulation of co-stimulatory molecules, the capacity to stimulate T cells in mixed leukocyte reaction and the ability to produce IL-12 and TNFα. They did not respond either to the bacterial stimulus LPS, a ligand for TLR-4, to the cytokine IFNγ, nor to any combination of LPS, IFNγ and anti-CD40 antibody.

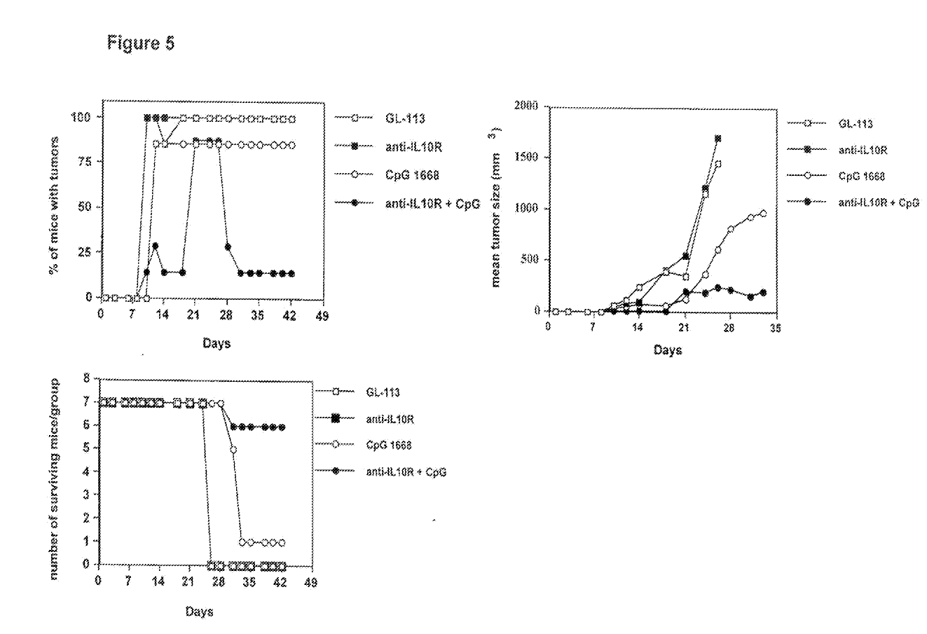

Therefore, it was hypothesized by the inventors that tumor-derived factors were inducing a refractory state in tumor-infiltrating DC, when considering the particular stimuli they used. Thus, elucidation of the factors that could inhibit this refractory state could lead to useful cancer therapeutics. In view of reports that IL-10, a DC inhibitory signal, is secreted by many human tumors (Chouaib et al., 1997, Immunol. Today 18:493-497; De Smedt et al., 1997, Eur. J. Immunol. 27:1229-1235; Caux et al., 1994, Int. Immunol. 6:1177-1185), the inventors tested whether antagonizing IL-10 could improve DC activation and therefore the host immune response against cancer. It was found, however, that treating mice with an antibody blocking IL-10 receptor (anti-IL10R) had little effect on the development of the C26 colon carcinoma tumor or its C26-6CK variant (the latter engineered as described in Vicari, et al., 2000, J. Immunol. 165:1992-2000 to express the chemokine CCL21/SLC/6Ckine: (See Example IV and FIG. 5)). Indeed, as shown in Examples II and III, an anti-IL10R antibody had no or minimal effect on the activation of tumor-infiltrating DC with the LPS+IFNγ+anti-CD40.

Subsequently, the inventors hypothesized that other activation signals, in particular signals mediated through pathogen-associated molecular pattern receptors of the toll-like family but distinct from TLR-4, could be operative in tumor-infiltrating dendritic cells. In particular, they studied the effect of CpG 1668, a ligand for TLR-9 in the mouse (Hemmi et al., 2000, Nature 408: 740-745). They observed, however, that CpG 1668 had marginal effect either in activating tumor-infiltrating dendritic cells (Examples II and III) or in the treatment of established subcutaneous tumors in mice (Examples V to VII).

In marked contrast, however, the inventors have surprisingly discovered that the combination of CpG 1668 and anti-IL10R induces IL-12p70 and TNFα production by C26-6CK tumor-infiltrating DC and greatly enhances the stimulatory capacity of those DC in MLR (See Examples II and III). Subsequently, the combination of CpG 1668 plus anti-IL10R antibody showed significant anti-tumor effect in mice bearing C26-6CK tumors (Example V). Furthermore, the combination of CpG 1668 and anti-IL10R antibody, but not the combination of LPS+IFNγ+anti-CD40 antibody was similarly able to induce IL-12 production in tumor-infiltrating DC from the parental C26 tumor and from tumors of other histological origin: the B16 melanoma and the LL2 lung carcinoma (See Example IV). The combination of CpG 1668 plus anti-IL10R also showed anti-tumor activity in the C26 and B16F0 tumor models (Examples VI and VII).

The invention therefore provides methods for treating cancer in a mammal comprising administering to said mammal an effective amount of a tumor-derived DC inhibitory factor antagonist in combination with an effective amount of a TLR agonist, through the activation of tumor-infiltrating dendritic cells.

A “tumor-derived dendritic cell (DC) inhibitory factor antagonist” as defined herein is an agent that is shown in a binding or functional assay to block the action of an agent which is secreted by tumor cells and is known to inhibit dendritic cell function.

A “TLR agonist” as defined herein is any molecule which activates a toll-like receptor (“TLR”) as described in Bauer et al., 2001, Proc. Natl. Acad. Sci. USA 98: 9237-9242. In a particularly preferred embodiment, the TLR agonist is an agonist of TLR9, such as described in Hemmi et al., 2000, Nature 408: 740-745 and Bauer et al., 2001, Proc. Natl. Acad. Sci. USA 98: 9237-9242.

### 1. Tumor-Derived DC Inhibitory Factor Antagonists

The term “tumor-derived DC inhibitory factor antagonists” includes any agent that blocks the action of a tumor-derived factor which induces a refractory state in tumor-infiltrating DC. Examples of such tumor-derived factors include, but are not limited to, IL-6, VEGF, CTLA-4, OX-40, TGF-β, prostaglandin, ganglioside, M-CSF, and IL-10 (Chouaib et al. 1997, Immunol. Today 18: 493-497).

Tumor-derived DC inhibitory factor antagonists may be identified by analyzing their effects on tumor dendritic cells in the presence of an activation stimulus. In the presence of an efficient amount of tumor-derived DC inhibitory factor antagonist, the tumor-dendritic cells would undergo a maturation process that can be followed by measuring the production of cytokines such as IL-12, TNFα, IFNα, or the expression of molecules typically expressed by mature dendritic cells such as CD80, CD86, CD83 and DC-Lamp. Alternatively, the effect of the tumor-derived DC inhibitory factor antagonist can be observed when analyzing the activation of human dendritic cells, not isolated from tumor, activated in the presence of purified or non-purified factors of tumor origin reported to inhibit dendritic cell maturation.

The tumor-derived DC inhibitory factor antagonists may act on the DC inhibitory factors themselves, as, for example, an anti-IL-10 monoclonal antibody would block the action of IL-10, or by any other means that would prevent the DC inhibitory factors from having their normal effect on tumor-infiltrating DC, as for example, an anti-IL-10R monoclonal antibody would prevent signaling of IL-10 through its receptor on DC.

Antagonists of tumor-derived DC inhibitory factors can be derived from antibodies or comprise antibody fragments. In addition, any small molecules antagonists, antisense nucleotide sequence, nucleotide sequences included in gene delivery vectors such as adenoviral or retroviral vectors that are shown in a binding or functional assay to inhibit the activation of the receptor would fall within this definition. It is well known in the art how to screen for small molecules which specifically bind a given target, for example tumor-associated molecules such as receptors. See, e.g., Meetings on High Throughput Screening, International Business Communications, Southborough, Mass. 01772-1749. Similarly, soluble forms of the receptor lacking the transmembrane domains can be used. Finally, mutant antagonist forms of the tumor-derived DC inhibitory factor can be used which bind strongly to the corresponding receptors but essentially lack biological activity.

In particularly preferred embodiments of the invention, the tumor-derived DC inhibitory factor antagonist is an IL-10 antagonist. The term “IL-10 antagonist” includes both antagonists of IL-10 itself and antagonists of the IL-10 receptor that inhibit the activity of IL-10. Examples of IL-10 antagonists which would be useful in this invention include, but are not limited to, those described in U.S. Pat. No. 5,231,012, issued Jul. 27, 1993 (directed to IL-10 and IL-10 antagonists) and U.S. Pat. No. 5,863,796, issued Jan. 26, 1999 (directed to the IL-10 receptor and IL-10 receptor antagonists), both of which are expressly incorporated herein by reference.

### 2. TLR Agonists

Several agonists of TLR derived from microbes have been described, such as lipopolysaccharides, peptidoglycans, flagellin and lipoteichoic acid (Aderem et al., 2000, Nature 406:782-787; Akira et al., 2001, Nat. Immunol. 2: 675-680) Some of these ligands can activate different dendritic cell subsets, that express distinct patterns of TLRs (Kadowaki et al., 2001, J. Exp. Med. 194: 863-869). Therefore, a TLR agonist could be any preparation of a microbial agent that possesses TLR agonist properties. For example, the penicillin-killed streptococcal agent OK-432 contains lipoteichoic acid which might induce the production of Th1 cytokines through TLR binding (Okamoto et al., 2000, Immunopharmacology 49: 363-376). Table 1 below lists several known TLR ligands:

TABLE 1
Known TLR ligands

| | | | | | | | | | |
|---|---|---|---|---|---|---|---|---|---|
| TLR1 | TLR2 | TLR3 | TLR4 | TLR5 | TLR6 | TLR7 | TLR8 | TLR9 | TLR10 |

| | | | | |
|---|---|---|---|---|
| | LTA | LPS | Flagellin | CpG |
| | PG | | | |
| TLR1 + TLR6 | TLR2 + TLR6 | | | |
| lipoprotein | lipoprotein | | | |

LTA: lipoteichoic acid
LPS: lipopolysacoharide
PG: peptidoglycan

Certain types of untranslated DNA have been shown to stimulate immune responses by activating TLRs. In particular, immunostimulatory oligonucleotides containing CpG motifs have been widely disclosed and reported to activate lymphocytes (See, e.g., U.S. Pat. No. 6,194,388). A “CpG motif” as used herein is defined as an unmethylated cytosine-guanine (CpG) dinucleotide. Immunostimulatory oligonucleotides which contain CpG motifs can also be used as TLR agonists according to the methods of the present invention.

Many immunostimulatory nucleotide sequences have been described in the art and may readily be identified using standard assays which indicate various aspects of the immune response, such as cytokine secretion, antibody production, NK cell activation and T cell proliferation. See, e.g. U.S. Pat. Nos. 6,194,388 and 6,207,646; WO 98/52962; WO 98/55495; WO 97/28259; WO 99/11275; Krieg et al., 1995, Nature 374:546-549; Yamamoto et al., 1992 J. Immunol. 148:4072-4076; Ballas et al., 1996, J. Immunol. 157(5) 1840-1845; Klinman et al., 1997, PNAS 93(7):2879-83; Shimada et al., 1986, Jpn. J. Cancer Res. 77:808-816; Cowdery et al., 1996, J. Immunol. 156:4570-75; Hartmann et al., 2000, J. Immunol. 164(3):1617-24.

The immunostimulatory nucleotide sequences can by of any length greater than 6 bases or base pairs. An immunostimulatory nucleotide sequence can contain modifications, such as modification of the 3′ OH or 5′ OH group, modifications of a nucleotide base, modifications of the sugar component, and modifications of the phosphate ring. The immunostimulatory nucleotide sequence may be single or double stranded DNA, as well as single or double-stranded RNA or other modified polynucleotides. An immunostimulatory nucleotide sequence may or may not include one or more palindromic regions.

The immunostimulatory nucleotide sequence can be isolated using conventional polynucleotide isolation procedures, or can be synthesized using techniques and nucleic acid synthesis equipment which are well known in the art including, but not limited to, enzymatic methods, chemical methods and the degradation of larger oligonucleotide sequences. (See, for example, Ausubel et al., 1987 and Sambrook et al., 1989).

Examples of immunostimulatory nucleotide sequences that are useful in the methods of the invention include but are not limited to those disclosed in U.S. Pat. No. 6,218,371; U.S. Pat. No. 6,194,388; U.S. Pat. No. 6,207,646; U.S. Pat. No. 6,239,116 and PCT Publication No. WO 00/06588 (University of Iowa); PCT Publication No. WO 01/62909; PCT Publication No. WO 01/62910; PCT Publication No. WO 01/12223; PCT Publication No. WO 98/55495; and PCT Publication No. WO 99/62923 (Dynavax Technologies Corporation), each of which is incorporated herein by reference.

In particular, U.S. Pat. No. 6,194,388 (University of Iowa) discloses immunostimulatory nucleic acids which comprise an oligonucleotide sequence including at least the following formula:

5′X₁X₂CGX₃X₄3′

wherein C and G are unmethylated, wherein X₁X₂ are dinucleotides selected from the group consisting of GpT, GpG, GpA, ApA, ApT, ApG, CpT, CpA, CpG, TpA, TpT, and TpG, and X₃X₄ are dinucleotides selected from the group consisting of: TpT, CpT, ApT, TpG, ApG, CpG, TpC, ApC, CpC, TpA, ApA and CpA and wherein at least one nucleotide has a phosphate backbone modification. For facilitating uptake into cells, preferred CpG containing immunostimulatory oligonucleotides are described as being in the range of 8 to 40 base pairs in size. Immunostimulatory oligonucleotides that fall within this formula would be useful in the presently claimed methods.

WO 99/62923 discloses additional examples of immunostimulatory nucleotide sequences that may be used in conjunction with the present invention. In particular, modified immunostimulatory nucleotide sequences comprising hexameric sequences or hexanucleotides comprising a central CG sequence, where the C residue is modified by the addition to C-5 and/or C-6 with an electron-withdrawing moiety are disclosed.

Immunostimulatory oligonucleotides can be stabilized by structure modification which renders them relatively resistant to in vivo degradation. Examples of stabilizing modifications include phosphorothioate modification (i.e., at least one of the phosphate oxygens is replaced by sulfur), nonionic DNA analogs, such as alkyl- and aryl-phosphonates (in which the charged phosphonate oxygen is replaced by an alkyl or aryl group), phosphodiester and alkylphosphotriesters, in which the charged oxygen moiety is alkylated. Oligonucleotides which contain a diol, such as tetraethyleneglycol or hexaethyleneglycol, at either or both termini have also been shown to be substantially resistant to nuclease degradation (See U.S. Pat. No. 6,194,388 (University of Iowa)).

The immunostimulatory nucleotide sequences may also be encapsulated in or bound to a delivery complex which results in higher affinity binding to target cell surfaces and/or increased cellular uptake by target cells. Examples of immunostimulatory nucleotide sequence delivery complexes include association with a sterol (e.g. cholesterol), a lipid (e.g. a cationic lipid, virosome or liposome), or a target cell specific binding agent (e/g/a ligand recognized by target cell specific receptor). Preferred complexes must be sufficiently stable in vivo to prevent significant uncoupling prior to internalization by the target cell. However, the complex should be cleavable under appropriate conditions within the cell so that the oligonucleotide is released in a functional form (U.S. Pat. No. 6,194,388; WO 99/62923).

In a particularly preferred embodiment, the TLR agonist is an agonist of TLR9, such as described in Hemmi et al., 2000, Nature 408: 740-745 and Bauer et al., 2001, Proc. Natl. Acad. Sci. USA 98: 9237-9242. The known ligands for TLR-9, to date, are unmethylated oligonucleotide sequences containing CpG motifs such as CpG 1668 in the mouse (TCCATGACGTTCCTGATGCT) (SEQ ID NO: 5) and CpG 2006 in man (TCGTCGTTTTGTCGTTTTGTCGTT) (SEQ ID NO: 1) (Bauer et al., 2001, Proc. Natl. Acad. Sci. USA 98: 9237-9242). Table 2 below lists preferred agonists of TLR9:

TABLE 2

Examples of CpG active on human DC:

| | | |
|---|---|---|
| CpG 2006: | TCGTCGTTTGTCGTTTTGTCGTT | (SEQ ID NO: 1) |
| CPG 2216: | GGGGGACGATCGTCGGGGGG | (SEQ ID NO: 2) |
| AAC-30: | ACCGATAACGTTGCCGGTGACGGCACCACG | (SEQ ID NO: 3) |
| GAC-30: | ACCGATGACGTCGCCGGTGACGGCACCACG | (SEQ ID NO: 4) |

In addition to those mentioned above, ligand screening using TLRs or fragments thereof can be performed to identify other molecules, including small molecules having binding affinity to the receptors. See, e.g., Meetings on High Throughput Screening, International Business Communications, Southborough, Mass. 01772-1749. Subsequent biological assays can then be utilized to determine if a putative agonist can provide activity. If a compound has intrinsic stimulating activity, it can activate the receptor and is thus an agonist in that it stimulates the activity of ligand, e.g., inducing signaling.

An “effective amount” of a TLR agonist as used herein is an amount which elicits the desired biological effect. In particular, an effective amount is that amount which, when combined with an effective amount of a tumor-derived DC inhibitory factor antagonist, is sufficient to trigger the activation of tumor-infiltrating DC.

An “effective amount” of a tumor-derived DC inhibitory factor antagonist is an amount which elicits the desired biological effect. In particular, an effective amount is that amount which, when combined with an effective amount of a TLR agonist, is sufficient to trigger the activation of tumor-infiltrating DC.

Administration “in combination” refers to both simultaneous and sequential administration. The tumor-derived DC inhibitory factor antagonists can be delivered or administered at the same site or a different site and can be administered at the same time or after a delay not exceeding 48 hours. Concurrent or combined administration, as used herein, means that the tumor-derived DC inhibitory factor antagonist and/or TLR agonist and/or antigen are administered to the subject either (a) simultaneously, or (b) at different times during the course of a common treatment schedule. In the latter case, the two compounds are administered sufficiently close in time to achieve the intended effect.

The tumor-derived DC inhibitory factor antagonists and/or TLR agonists used in practicing the invention may be recombinant protein with an amino-acid sequence identical to the natural product, or a recombinant protein derived from the natural product but including modifications that changes its pharmacokinetic properties and/or add novel biological properties while keeping its original DC activating or antitumor properties.

The mode of delivery of the tumor-derived DC inhibitory factor antagonist and/or TLR agonist may be by injection, including intravenously, intratumorally, intradermally, intramuscularly, subcutaneously, or topically.

In a particularly preferred embodiment of the invention, the tumor-derived DC inhibitory factor antagonist(s) and TLR agonist(s) are administered in combination with a tumor-associated antigen. Tumor associated antigens for use in the invention include, but are not limited to Melan-A, tyrosinase, p97, β-HCG, GalNAc, MAGE-1, MAGE-2, MAGE-3, MAGE-4, MAGE-12, MART-1, MUC1, MUC2, MUC3, MUC4, MUC18, CEA, DDC, melanoma antigen gp75, HKer 8, high molecular weight melanoma antigen, K19, Tyr1 and Tyr2, members of the pMel 17 gene family, c-Met, PSA, PSM, α-fetoprotein, thyroperoxidase, gp100, NY-ESO-1, telomerase and p53. This list is not intended to be exhaustive, but merely exemplary of the types of antigen which may be used in the practice of the invention.

Other antigens different from tumor-associated antigens may be administered together with the tumor-derived DC inhibitory factor antagonist(s) and TLR agonist(s) in order to increase the specific immune response against these antigens. These antigens include but are not restricted to native or modified molecules expressed by bacteria, viruses, fungi, parasites. The antigens may also include allergens and auto-antigens, and in this case the combination of the tumor-derived DC inhibitory factor antagonist(s) and TLR agonist(s) will be administered in conjunction with the antigen in order to re-direct the immune response towards a more favorable outcome, e.g. to transform a Th2-type immune response into a Th1-type immune response.

Different combinations of antigens may be used that show optimal function with different ethnic groups, sex, geographic distributions, and stage of disease. In one embodiment of the invention at least two or more different antigens are administered in conjunction with the administration of the tumor-derived DC inhibitory factor antagonist(s) and TLR agonist(s) combination.

The tumor-derived DC inhibitory factor antagonist and/or TLR agonist may be administered in combination with each other and/or with the antigen(s) or may be linked to each other or to the antigen(s) in a variety of ways (see, for example, WO 98/16247; WO 98/55495; WO 99/62823). For example, TLR agonist and/or a tumor-derived DC inhibitory factor and/or an antigen may be administered spatially proximate with respect to each other, or as an admixture (i.e. in solution). Linkage can be accomplished in a number of ways, including conjugation, encapsidation, via affixation to a platform or adsorption onto a surface.

To conjugate TLR agonist(s) to tumor-derived DC inhibitory factor antagonist(s) and/or antigen(s), a variety of methods may be used. The association can be through covalent interactions and/or through non-covalent interactions, including high affinity and/or low affinity interactions. Examples of non-covalent interactions that can couple a TLR agonist and a tumor-derived DC inhibitory factor include, but are not limited to, ionic bonds, hydrophobic interactions, hydrogen bonds and van der Walls attractions. When the tumor-derived DC inhibitory factor antagonist is a protein or antibody and the TLR agonist is an immunostimulatory polynucleotide, for example, the peptide portion of the conjugate can be attached to the 3′-end of the immunostimulatory polynucleotide through solid support chemistry using methods well-known in the art (see, e.g., Haralambidis et al., 1990a, Nucleic Acids Res. 18:493-499 and Haralambidis et al., 1990b, Nucleic Acids Res. 18:501-505). Alternately, the incorporation of a “linker arm” possessing a latent reactive functionality, such as an amine or carboxyl group, at C-5 of a cytosine base provides a handle for the peptide linkage (Ruth, 4^{th} Annual Congress for Recombinant DNA Research, p. 123). The linkage of the immunostimulatory polynucleotide to a peptide can also be formed through a high-affinity, non-covalent interaction such as a biotin-streptavidin complex. A biotinyl group can be attached, for example, to a modified base of an oligonucleotide (Roget et al., Nucleic Acids Res. (1989) 17:7643-7651). Incorporation of a streptavidin moiety into the peptide protion allows formation of a non-covalently bound complex of the streptavidin conjugated peptide and the biotinylated polynucleotide.

A moiety designed to further activate or stimulate maturity of the DC may be advantageously administered. Examples of such agents are TNF-α, IFN-α, RANK-L or agonists of RANK, CD40-L or agonists of CD40 Such activating agents can provide additional maturation signals which can participate, in conjunction with the TLR agonist(s) i) in driving the migration of DC from tissues toward lymphoid organs through the draining lymph, and ii) in activating DC to secrete molecules which enhance immune responses—in particular the anti-tumor response—such as IL-12 and IFNα (Banchereau et al. 1998, Nature 392: 245-252).

GM-CSF, G-CSF or FLT3-L can also advantageously be administered in the methods of the invention. GM-CSF, G-CSF or FLT3-L may be administered for purposes of increasing the number of circulating DC which might then be locally recruited locally in the tumor. This protocol would imply a systemic pre-treatment for a least five to seven days with GM-CSF, G-CSF or FLT3-L. An alternative would be to favor by local administration of GM-CSF, G-CSF or FLT3-L the local differentiation of DC-precursors (monocytes, plasmacytoid precursors of DC) into DC which could then pick up the antigen delivered at the same site.

In addition, chemokines or combinations of multiple chemokines may be advantageously administered in combination with the Tumor-derived DC inhibitory factor antagonists and TLR agonists of the invention. Chemokines which have been shown to have beneficial effects include CCL21, CCL3, CCL20, CCL16, CCL5, CCL25, CXCL12, CCL7, CCL8, CCL2, CCL13, CXCL9, CXCL10, CXCL11 (see, e.g., Sozzani et al., 1995, J. Immunol. 155:3292-3295; Sozzani et al., 1997, J. Immunol. 159: 1993-2000; Xu et al., 1996, J. Leukoc. Biol. 60; 365-371; MacPherson et al., 1995, J. Immunol. 154: 1317-1322; Roake et al., 1995, J. Exp. Med 181:2237-2247 and European Patent Application EP 0 974 357 A1 filed Jul. 16, 1998 and published Jan. 26, 2000). Generally, Tumor-derived DC inhibitory factor antagonists, TLR agonists and/or activating agent(s) and/or cytokine(s) are administered as pharmaceutical compositions comprising an effective amount of an Tumor-derived DC inhibitory factor antagonist and TLR agonist(s) and/or antigen(s) and/or activating agent(s) and/or cytokine(s) in a pharmaceutical carrier. These reagents can be combined for therapeutic use with additional active or inert ingredients, e.g., in conventional pharmaceutically acceptable carriers or diluents, e.g., immunogenic adjuvants, along with physiologically innocuous stabilizers and excipients. A pharmaceutical carrier can be any compatible, non-toxic substance suitable for delivering the compositions of the invention to a patient.

The cytokines and/or chemokines may optionally be delivered to the tumor using a targeting construct comprising a chemokine or cytokine or a biologically active fragment or variant thereof and a targeting moiety. A “targeting moiety” as referred to herein is a moiety which recognizes or targets a tumor-associated antigen or a structure specifically expressed by non-cancerous components of the tumor, such as the tumor vasculature. Examples of targeting moieties include but are not limited to peptides, proteins, small molecules, vectors, antibodies or antibody fragments which recognize or target tumor-associated antigens or structures specifically expressed by non-cancerous components of a tumor. In preferred embodiments, the targeting moiety is a peptide, a protein, a small molecule, a vector such as a viral vector, an antibody or an antibody fragment. In more preferred embodiments, the targeting moiety is an antibody or antibody fragment. In most preferred embodiments, the targeting vector is a ScFv fragment.

The targeting moiety can be specific for an antigen expressed by tumor cells, as it has been described in humans, for example, for the folate receptor (Melani et al., 1998, Cancer Res. 58: 4146-4154), Her2/neu receptor, Epidermal Growth Factor Receptor and CA125 tumor antigen (Glennie et al., 2000, Immunol. Today 21: 403-410). Several other tumor antigens can be used as targets and are either preferentially expressed, uniquely expressed, over-expressed or expressed under a mutated form by the malignant cells of the tumor (Boon et al., 1997, Curr. Opin. Immunol. 9: 681-683). These may include: Melan-A, tyrosinase, p97, —HCG, GalNAc, MAGE-1, MAGE-2, MAGE-3, MAGE-4, MAGE-12, MART-1, MUC1, MUC2, MUC3, MUC4, MUC18, CEA, DDC, melanoma antigen gp75, HKer 8, high molecular weight melanoma antigen, K19, Tyr1 and Tyr2, members of the pMel 17 gene family, c-Met, PSA, PSM, α-fetoprotein, thyroperoxidase, gp100, insulin-like growth factor receptor (IGF-R), telomerase and p53. This list is not intended to be exhaustive, but merely exemplary of the types of antigen which may be used in the practice of the invention. Alternatively, the targeting moiety can be specific for an antigen preferentially expressed by a component of the tumor different from the malignant cells, and in particular tumor blood vessels. The family of alpha v integrins, the VEGF receptor and the proteoglycan NG2 are examples of such tumor blood vessel-associated antigens (Pasqualini et al., 1997, Nat. Biotechnol. 15: 542-546).

Both primary and metastatic cancer can be treated in accordance with the invention. Types of cancers which can be treated include but are not limited to melanoma, breast, pancreatic, colon, lung, glioma, hepatocellular, endometrial, gastric, intestinal, renal, prostate, thyroid, ovarian, testicular, liver, head and neck, colorectal, esophagus, stomach, eye, bladder, glioblastoma, and metastatic carcinomas. The term “carcinoma” refers to malignancies of epithelial or endocrine tissues including respiratory system carcinomas, gastrointestinal system carcinomas, genitourinary system carcinomas, prostatic carcinomas, endocrine system carcinomas, and melanomas. Metastatic, as this term is used herein, is defined as the spread of tumor to a site distant to regional lymph nodes.

The quantities of reagents necessary for effective therapy will depend upon many different factors, including means of administration, target site, physiological state of the patient, and other medicants administered. Thus, treatment dosages should be titrated to optimize safety and efficacy. Animal testing of effective doses for treatment of particular cancers will provide further predictive indication of human dosage. Various considerations are described, e.g., in Gilman et al. (eds.) (1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa. Methods for administration are discussed therein and below, e.g., for intravenous, intraperitoneal, or intramuscular administration, transdermal diffusion, and others. Pharmaceutically acceptable carriers will include water, saline, buffers, and other compounds described, e.g., in the Merck Index, Merck & Co., Rahway, N.J. Slow release formulations, or a slow release apparatus may be used for continuous administration.

Dosage ranges for tumor-derived DC inhibitory factor antagonists and/or TLR agonists agent(s) will vary depending on the form of the agonist/antagonists. For example, the effective dose of an IL-10 receptor antibody typically will range from about 0.05 to about 25 μg/kg/day, preferably from about 0.1 to about 20 μg/kg/day, most preferably from about 1 to about 10 μg/kg/day. For immunogenic compositions such as TLR agonists, the amounts can vary based on the form of the TLR agonist, the individual, what condition is to be treated and other factors evident to one skilled in the art. Factors to be considered include the antigenicity, whether or not the TLR agonist will be complexed or covalently attached to an adjuvant or delivery molecule, route of administration and the number of immunizing doses to be administered. Such factors are known in the art. A suitable dosage range is one that provides the desired activation of dendritic cells. Generally, a dosage range for an immunostimulatory oligonucleotide may be, for example, from about any of the following: 0.1 to 100 μg, 0.1 to 50 μg, 0.1 to 25 μg, 0.1 to 10 μg, 1 to 500 μg, 100 to 400 μg, 200 to 300 μg, 1 to 100 μg, 100 to 200 μg, 300 to 400 μg, 400 to 500 μg. Alternatively, the doses can be about any of the following: 0.1 μg, 0.25 μg, 0.5 μg, 1.0 μg, 2.0 μg, 5.0 μg, 10 μg, 25 μg, 50 μg, 75 μg, 100 μg. Accordingly, dose ranges can be those with a lower limit about any of the following: 0.1 μg, 0.25 μg, 0.5 μg and 1.0 μg; and with an upper limit of about any of the following: 25 μg, 50 μg and 100 μg. In these compositions, the molar ratio of ISS-containing polynucleotide to antigen may vary. The absolute amount given to each patient depends on pharmacological properties such as bioavailability, clearance rate and route of administration.

Generally, treatment is initiated with smaller dosages which are less than the optimum dose of the compound. Thereafter, the dosage is increased by small increments until the optimum effect under the circumstance is reached. Determination of the proper dosage and administration regime for a particular situation is within the skill of the art.

Dosage of tumor-derived DC inhibitory factor antagonists and TLR agonists which are administered by means of a vector will largely depend on the efficacy of the particular vector employed and the condition of the patient, as well as the body weight or surface area of the patient to be treated. The size of the dose also will be determined by the existence, nature, and extent of any adverse side-effects that accompany the administration of a particular vector, or transduced cell type in a particular patient. In determining the effective amount of the vector to be administered in the treatment, the physician evaluates circulating plasma levels of the vector, vector toxicities, progression of the disease, and the production of anti-vector antibodies. The typical dose for a nucleic acid is highly dependent on route of administration and gene delivery system. Depending on delivery method the dosage can easily range from about 1 μg to 100 mg or more. In general, the dose equivalent of a naked nucleic acid from a vector is from about 1 μg to 100 μg for a typical 70 kilogram patient, and doses of vectors which include a viral particle are calculated to yield an equivalent amount of therapeutic nucleic acid.

The preferred biologically active dose of GM-CSF, G-CSF or FLT-L in the practice of the claimed invention is that dosing combination which will induce maximum increase in the number of circulating CD14⁺/CD13⁺ precursor cells; the expression of antigen presenting molecules on the surface of DC precursors and mature DC; antigen presenting activity to T cells; and/or stimulation of antigen-dependent T cell response consistent with mature DC function. The amount of GM-CSF to be used for subcutaneous administration typically ranges from about 0.25 μg/kg/day to about 10.0 μg/kg/day, preferably from about 1.0-8.0 μg/kg/day, most preferably 2.5-5.0 μg/kg/day. An effective amount for a particular patient can be established by measuring a significant change in one or more of the parameters indicated above.)

### EXAMPLES

The invention can be illustrated by way of the following non-limiting examples.

### Example I

### C26-6CK Tumor-Infiltrating Dendritic Cells are Unresponsive to the Combination of LPS+Anti-CD40+IFNγ when Compared to Bone Marrow-Derived Dendritic Cells

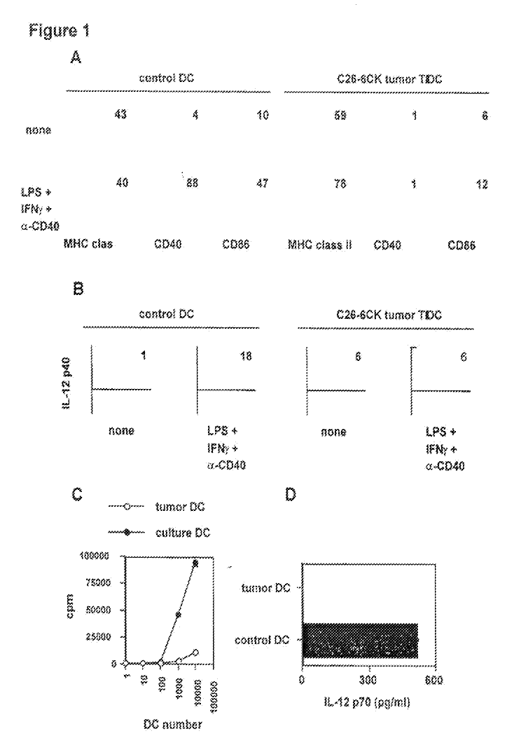

In this example, the inventors have shown that DC infiltrating C26-6CK tumors do not respond to LPS+IFNγ+anti-CD40 antibody when considering IL-12 production or stimulatory capacity in mixed leukocyte reaction (MLR), in comparison with bone marrow-derived DC (FIG. 1). All tumor cell cultures were performed in DMEM (Gibco-BRL, Life Technologies, Paisley Park, Scotland) supplemented with 10% FCS (Gibco-BRL), 1 mM hepes (Gibco-BRL), Gentallin (Schering-Plough, Union, N.J.), 2×10⁻⁵ M beta-2 mercaptoethanol (Sigma, St Louis, Mo.). All cell cultures were performed at 37° C. in a humidified incubator with 5% CO₂. The C26 colon carcinoma and TSA mammary carcinoma were provided by Mario Colombo (Istituto Nazionale per lo Studio e la Cura dei Tumori, Milano, Italy). The B16F0 melanoma and LL2 lung carcinoma were obtained from American Type Culture Collection (LGC, Strasbourg, France). The C26-6CK cell line engineered to stably secrete the mouse chemokine 6Ckine/SLC/CCL21 has been described previously by the inventors (Vicari et al., 2000, J. Immunol. 165: 1992-2000) TIDC from C26-6CK tumors were enriched using anti-CD11c magnetic beads (Myltenyi Biotec Gmbh, Germany). Bone marrow-derived DCs were obtained by culture of bone marrow progenitors with GM-CSF (Schering-Plough, Union, N.J.) and TNFα (R&D Systems, Minneapolis, Minn.) for 5 days. Activation was performed overnight by adding 10 ng/ml LPS (Sigma, St Louis, Mo.), 20 ng/ml IFNγ (R&D Systems) and 20 μg/ml purified FKG45.5 agonist anti-CD40 antibody (a kind gift from AG Rolink, Basel Institute for Immunology, Switzerland) to culture medium. FIG. 1A shows analysis of surface expression of MHC class II, CD40 and CD86 by FACS (gated on CD11c positive cells) FIG. 1B depicts Intracellular expression of IL-12p40 by CD11c+ cells after 20 hours, including 2.5 hour incubation with Brefeldin A. In FIG. 1C, mixed leukocyte reaction TIDC or bone marrow-derived DC stimulated with LPS+IFNγ+anti-CD40 were irradiated and cultured for 5 days at varying numbers in the presence of a constant number of enriched allogeneic T cells (3×10⁵ T cells). Proliferation was measured during the last 18 hours of culture by radioactive thymidine incorporation. FIG. 1D depicts measurement of IL-12 p70 in culture supernatants after activation with LPS+IFNγ+anti-CD40 by a specific ELISA.

These combined results suggest that dendritic cells infiltrating C26-6CK tumors are not able to acquire typical functions of dendritic cells upon stimulation with the combination of LPS+IFNγ+anti-CD40, namely the capacity to stimulate allogeneic T cells and the ability to secrete IL-12. These impaired functions are likely to be the results of the interaction of dendritic cells with tumors.

### Example II

### CpG 1668+Anti-IL10R Combination Restored IL-12 and TNFα in C26-6CK Tumor-Infiltrating Dendritic Cells

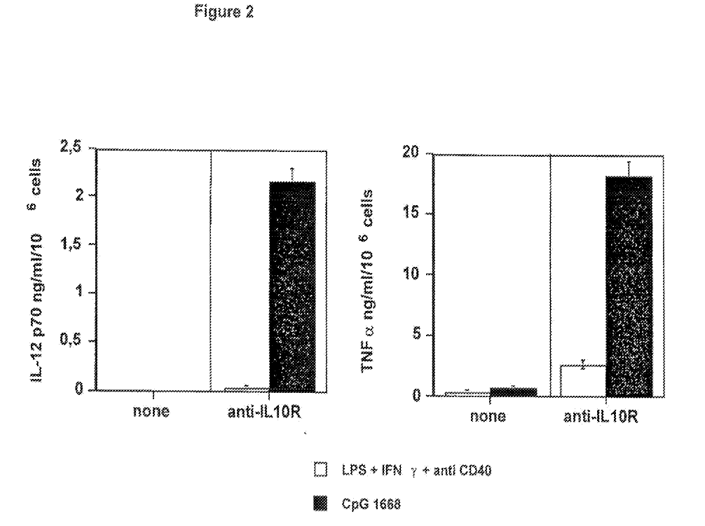

In this example, the inventors have shown that combined administration of CpG 1668 and anti-IL10R antibody restored IL-12 and TNFα in C26-6CK tumor-infiltrating dendritic cells (FIG. 2).

TIDC from C26-6CK tumors were enriched using anti-CD11c magnetic beads. Activation was performed overnight in the presence of GM-CSF 10 ng/ml. Activators were used at: 10 ng/ml LPS, 20 ng/ml IFNγ, 20 μg/ml FKG45.5 agonist anti-CD40 antibody, 5 μg/ml CpG 1668 (sequence: TCC-ATG-ACG-TTC-CTG-ATG-CT, phosphorothioate modified, MWG Biotech, Germany) and 10 μg/ml anti-IL10R (clone 1B13A, Castro et al., 2000, J. Exp. Med. 192: 1529-1534). IL-12 p70 and TNFα were measured in culture supernatants after 24 h stimulation using specific ELISAs.

Overall, these results indicate that CpG 1668 by itself does not induce IL-12 production by C26-6CK tumor-infiltrating DC. Anti-IL10R have either no effect by itself (not shown) or minimal effect when combined with LPS+IFNγ+anti-CD40. Only the combination of anti-IL10R and CpG 1668 was able to induce a significant production of bioactive II-12 and TNFα from C26-6CK tumor-infiltrating DC.

### Example III

### CpG 1668+Anti-IL10R Combination Restored MLR Stimulatory Capacity in C26-6CK Tumor-Infiltrating Dendritic Cells

In this example, the inventors have shown that combined administration of CpG 1668 and anti-IL-10 receptor antibody restored MLR stimulatory capacity.

TIDC from C26-6CK tumors were enriched using anti-CD11c magnetic beads and cultured overnight in the presence of GM-CSF and various combinations of LPS, IFNγanti-CD40, anti-IL10R and CpG 1668. Cells were then irradiated and cultured for 5 days at varying numbers in the presence of a constant number of enriched allogeneic T cells (3×10⁵ T cells). Proliferation was measured during the last 18 hours of culture by radioactive thymidine incorporation. The results show that tumor-infiltrating DC are poor stimulator cells in the MLR assay, but that their stimulatory capacity can be minimally enhanced with CpG1668, further enhanced with the combination of anti-IL10R+LPS+IFNγ+anti-CD40, and best enhanced with the combination of anti-IL10R and CpG 1668. Thus, this example shows that anti-IL10R plus CpG 1668 is the most suitable combination to restore DC stimulatory capacity in MLR. This could translate into a better priming of naive T cells in vivo, and therefore to a better T cell-mediated immune response against tumors when using the combination of an IL-10 antagonist and a TLR9 agonist to treat cancer.

### Example IV

### Tumor-Infiltrating Dendritic Cells from C26 Wild-Type and Tumors from Other Histological Nature are Unresponsive to LPS+IFNγ+Anti-CD40 but Produce IL-12 in Response to CpG 1668+Anti-IL10R

This example shows that tumor-infiltrating dendritic cells from C26 wild-type and tumors from other histological nature are unresponsive to LPS+IFNγ+anti-CD40 but produce IL-12 in response to CpG 1668+anti-IL10R.

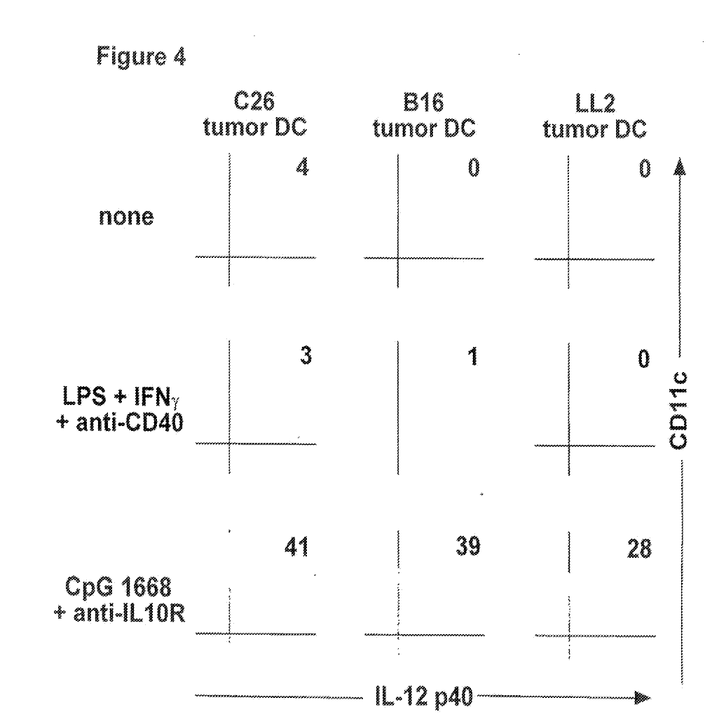

TIDC from C26 colon carcinoma, B16 melanoma and LL2 lung carcinoma tumors, all grown subcutaneously, were enriched using anti-CD11c magnetic beads and cultured overnight in the presence of GM-CSF and various combinations of LPS, IFNγ anti-CD40, anti-IL10R and CpG 1668. FACS analysis of intracellular expression of IL-12p40 versus surface expression of CD11c after 20 hours, including 2.5 hour incubation with Brefeldin A. FIG. 4 shows that, as found for the C26-6CK tumors, DC isolated from parental C26 tumors as well as tumors of different histological origin are not responsive to activation with LPS, IFNγ anti-CD40 but do respond to the combination of the TLR-9 agonist CpG 1668 plus anti-IL10R by producing IL-12. Thus, these observations suggest that the combination of an IL10 antagonist and a TLR-9 agonist could be an effective therapy in a variety of tumors.

### Example V

### Therapeutic Effect of CpG1668+Anti-IL10R Antibody in the C26-6CK Tumor Model

1×10⁵ C26-6CK tumor cells were implanted s.c. at Day 0 in groups of seven 8 week-old female BALB/c mice and treated as follow:
10 μg of CpG 1668 were injected peri- (when tumor too small) or intratumorally at Day 7, 14, and 21.
250 μg anti-IL10R purified antibody were injected intraperitoneally twice a week starting at Day 7 (stop Day 24). Control antibody was purified GL113 antibody.

Tumor development was assessed three times a week by palpation and tumors measured using a caliper with tumor volume=I²×L×0.4, I being the small diameter and L the large diameter. Mice were sacrificed when tumors exceeded 1500 mm³ or for humane criteria.

FIG. 5 shows that all mice injected with control antibody or anti-IL10R antibody alone developed tumors within 7 to 10 days, that eventually led to the sacrifice of animals at around 4 weeks. Injection of the TLR-9 agonist CpG 1668 had a minor effect since 1/7 mouse did not develop a tumor. In addition, survival was slightly better in this CpG 1668 group and the mean volume of tumors smaller than in the control group after three weeks. In contrast, mice treated with the combination of CpG 1668 and anti-IL10R, although developing palpable tumors, rejected these tumors for 6 out of 7 mice. Subsequently, those mice remained tumor-free for the rest of the experiment. These results indicate that the combination of TLR-9 agonist and IL-10 antagonist has therapeutic value in the C26-6CK model, suggesting that it could be used to treat other tumors, including in man.

### Example VI

### Therapeutic Effect of CpG 1668+Anti-IL10R Antibody in the C26 Tumor Model

5×10⁴ C26 tumor cells were implanted s.c. at Day 0 in groups of seven 8 week-old female BALB/c mice and treated as follow:
5 μg of CpG 1668 were injected intra-tumorally at Day 7, 14, and 21.
250 μg anti-IL10R purified antibody were injected intraperitoneally at Day 7, 14, and 21. Control antibody was purified GL113 antibody.

Tumor development was assessed three times a week by palpation and tumors measured using a caliper with tumor volume=I²×L×0.4, I being the small diameter and L the large diameter. Mice were sacrificed when tumors exceeded 1500 mm³ or for humane criteria.

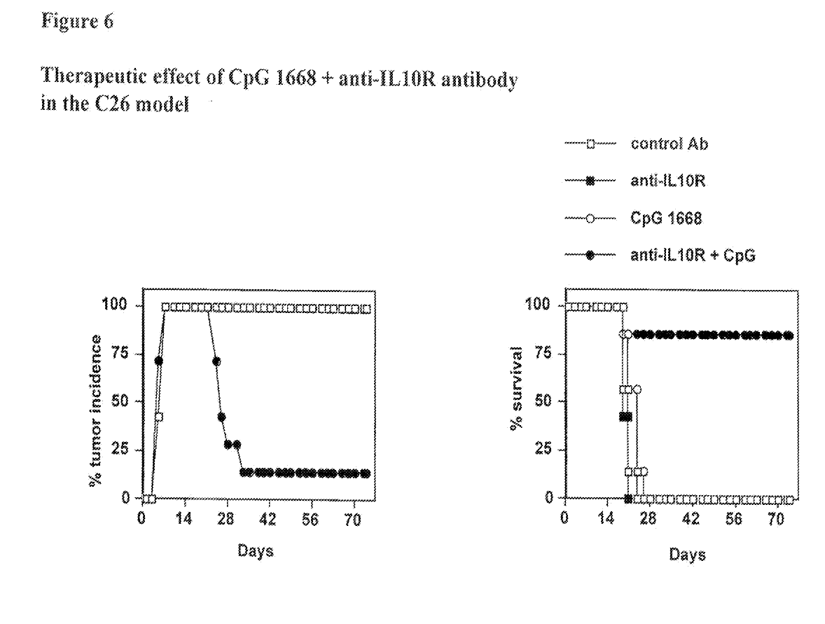

FIG. 6 shows that all mice injected with control antibody, CpG1668 or anti-IL10R antibody alone developed tumors within 7 days, that eventually led to the sacrifice of animals at around 3 to 4 weeks. In contrast, mice treated with the combination of CpG 1668 and anti-IL10R, although developing palpable tumors, rejected these tumors for 6 out of 7 mice. Subsequently, those mice remained tumor-free for the rest of the experiment. These results indicate that the combination of TLR-9 agonist and IL-10 antagonist has therapeutic value in the C26 model, suggesting that it could be used to treat other tumors, including in man.

### Example VII

### Therapeutic Effect of CpG 1668+Anti-IL10R Antibody in the B16F0 Melanoma Tumor Model

5×10⁴ B16F0 tumor cells were implanted s.c. at Day 0 in groups of seven 8 week-old female C57BL/6 mice and treated as follow:
5 μg of CpG 1668 were injected intra-tumorally at Day 7, 14, and 21.
250 μg anti-IL10R purified antibody were injected intraperitoneally at Day 7, 14, and 21. Control antibody was purified GL113 antibody.

Tumor development was assessed three times a week by palpation and tumors measured using a caliper with tumor volume=I²×L×0.4, I being the small diameter and L the large diameter. Mice were sacrificed when tumors exceeded 1500 mm³ or for humane criteria.

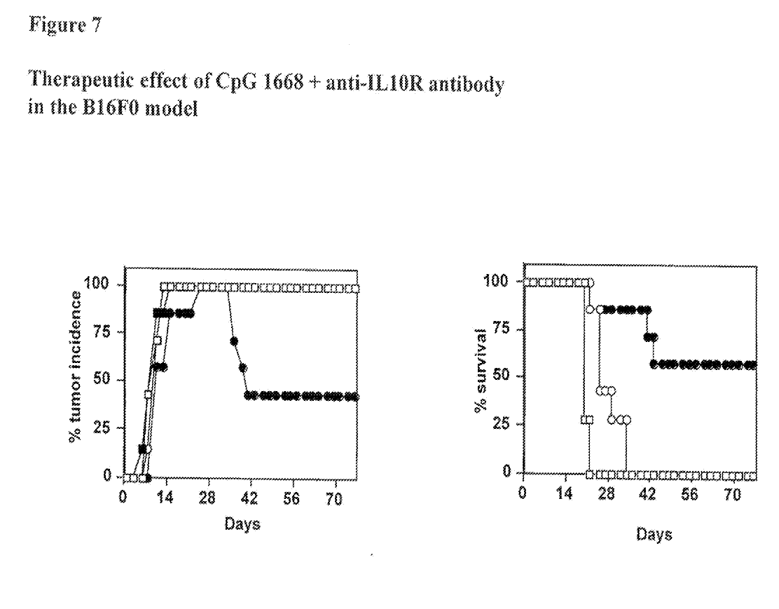

FIG. 7 shows that all mice injected with control antibody, CpG1668 or anti-IL10R antibody alone developed tumors within 7 days, that eventually led to the sacrifice of animals at around 3 to 4 weeks. CpG 1668 alone had a minor effect on survival. In contrast, mice treated with the combination of CpG 1668 and anti-IL10R, although developing palpable tumors, rejected these tumors for 6 out of 7 mice. Subsequently, those mice remained tumor-free for the rest of the experiment. These results indicate that the combination of TLR-9 agonist and IL-10 antagonist has therapeutic value in the B16F0 model, suggesting that it could be used to treat other tumors, including in man.

### Example VIII

### Tumor-Infiltrating DC from C26-6CK Tumors can Produce IL-12 in Response to the Combination of Anti-IL10 Antibody and CpG 1668

TIDC from C26-6CK tumors were enriched using anti-CD11c magnetic beads and cultured overnight in the presence of GM-CSF and various combinations of an anti-IL10 purified antibody and CpG 1668. FACS analysis of intracellular expression of IL-12p40 versus surface expression of CD11c after 20 hours, including 2.5 hour incubation with Brefeldin A.

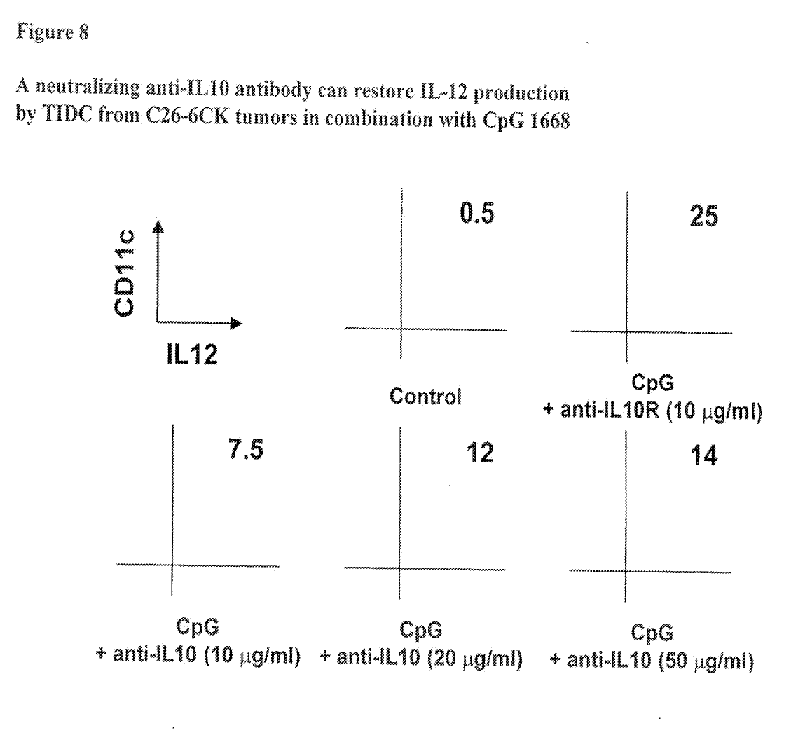

FIG. 8 shows that the combination of CpG 1668 and anti-IL10 can induce IL-12 production in C26-6CK tumor-infiltrating dendritic cells, suggesting that an antagonist of IL-10 itself, when associated with an effective amount of TLR-9 agonist, is effective in the treatment of cancer.

### Example IX

### The Inhibition of Bone-Marrow Derived DC Activation by a Supernatant from a C26 Tumor can be Restored by Anti-IL10R and/or Indomethacin, an Inhibitor of Cyclo-Oxygenases

Bone marrow-derived DCs were obtained by culture of bone marrow progenitors with GM-CSF and TNFα for 5 days in the presence or absence of 10% v/v of a supernatant from C26 tumors. To prepare tumor supernatant, 0.5 cm C26 tumors grown subcutaneously in BALB/c mice were excised and minced, then cultured for 48 hours in 10 ml DMEM. The resulting supernatant was filtered at 0.2 μm and frozen before use. This supernatant contained 0.25 ng/ml IL-10 and 50 ng/ml PGE₂, as determined by specific ELISA (R&D Systems). In order to inhibit PGE2 synthesis in the supernanant, the inhibitor of cyclo-oxygenase indomethacin (Sigma) was added at 1 μg/ml during the 48 h culture.

After 5 days, bone-marrow DC were activated with different combinations of optimal doses of LPS, IFNγ and anti-CD40 antibody in the presence or absence of 10 μg/ml anti-IL10R antibody. The activation of DC was measured by their expression of the co-stimulatory molecules CD40 and CD86 by FACS as well as by the production of IL-12 as detected by intra-cellular staining.

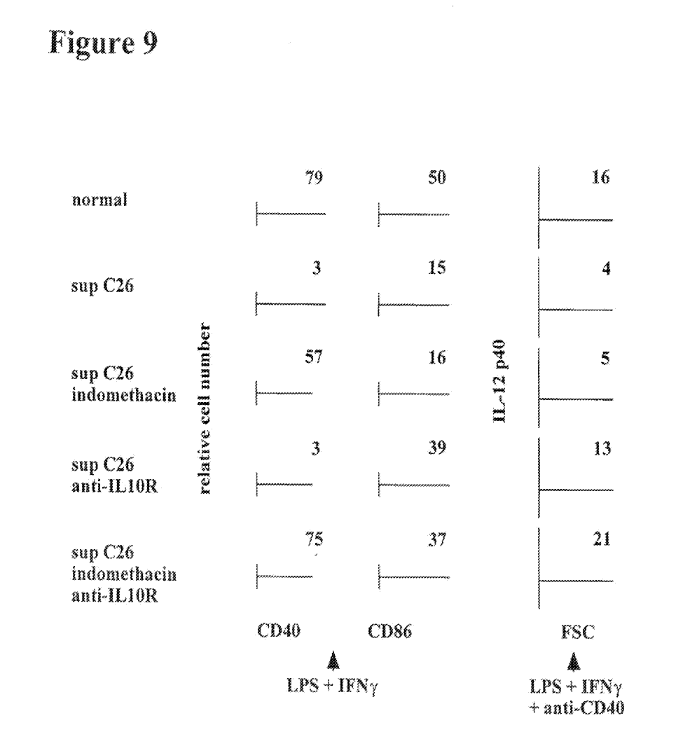

FIG. 9 shows that the C26 tumor supernatant is able to inhibit DC activation. Addition of a supernatant made in the presence of indomethacin or of anti-IL10R to the DC culture relieved partially the effect, while the combination of both could fully restore the up-regulation of CD40 and CD86 as well as IL-12 expression.

These experiments strongly suggest that products of cyclo-oxygenases, in particular prostaglandins, are also tumor-derived DC inhibitory factor.

### Example X

### Therapeutic Effect of CpG 1668+Indomethacin in the C26-6CK Tumor Model

5×10⁴ C26-6CK tumor cells were implanted s.c. at Day 0 in groups of seven 6 week-old female BALB/c mice and treated as follow:
5 μg of CpG 1668 were injected intra-tumorally at Day 7, 14, and 21.
5 μg/ml of indomethacin ad libitum in drinking water starting at day 5 until day 28

Tumor development was assessed three times a week by palpation. Mice were sacrificed when tumors exceeded 1500 mm³ or for humane criteria.

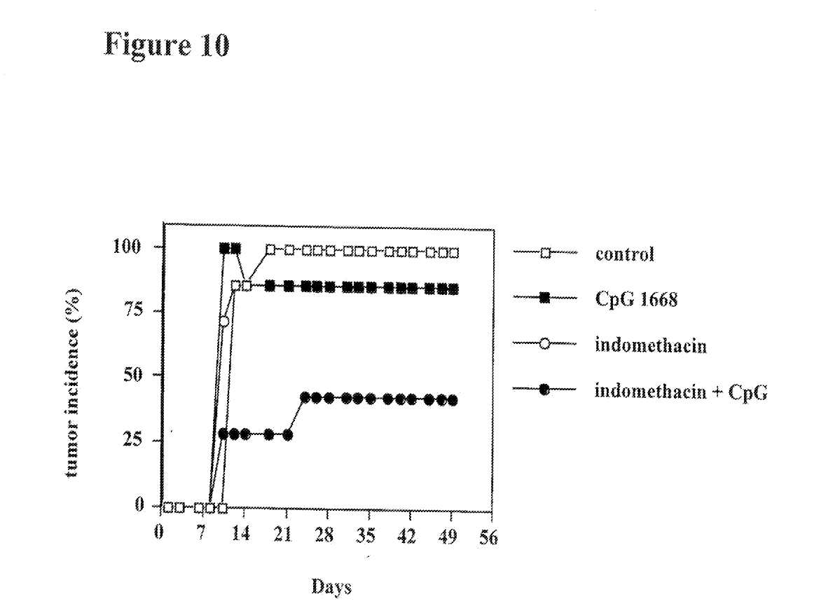

FIG. 10 shows that all control mice developed tumors within 7 days, that eventually led to the sacrifice of animals at around 3 to 4 weeks. Only 1/7 mouse in the CpG or indomethacin groups did not develop tumor. In contrast, 4/7 mice treated with the combination of CpG 1668 and indomethacin did not develop tumor. These results indicate that the combination of TLR-9 agonist and inhibitor of cyclo-oxygenase has therapeutic value in the C26-6CK model, suggesting that it could be used to treat other tumors, including in man.

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A method of treating cancer comprising administering to an individual in need thereof an effective amount of a tumor-derived dendritic cell (DC) inhibitory factor antagonist in combination with an effective amount of a TLR agonist.

2. The method of claim 1 wherein the tumor-derived DC inhibitory factor antagonist is selected from the group consisting of an IL-6 antagonist, a VEGF antagonist, a CTLA-4 antagonist, an OX-40 antagonist, a TGF-B antagonist, a prostaglandin antagonist, a ganglioside antagonist, an M-CSF antagonist, and an IL-10 antagonist.

3. The method of claim 2 wherein the tumor-derived DC inhibitory factor antagonist is an IL-10 antagonist.

4. The method of claim 3 wherein the IL-10 antagonist is selected from the group consisting of an antagonist of IL-10 and an antagonist of the IL-10 receptor.

5. The method of claim 4 wherein the IL-10 antagonist is:
a) recombinant;
b) a natural ligand;
c) a small molecule;
d) an antibody or antibody fragment;
e) an antisense nucleotide sequence; or
f) a soluble IL-10 receptor molecule.

6. The method of claim 5 wherein the antibody is a monoclonal antibody.

7. The method of claim 6 wherein the antibody is an anti-IL-10R monoclonal antibody.

8. The method of claim 1 wherein the TLR agonist is:
a) recombinant;
b) a natural ligand;
a) an immunostimulatory nucleotide sequence;
b) a small molecule;
c) a purified bacterial extract;
d) an inactivated bacteria preparation.

9. The method of claim 1 wherein the TLR agonist is an agonist of TLR-9.

10. The method of claim 9 wherein the TLR agonist is an immunostimulatory nucleotide sequence.

11. The method of claim 10 wherein the immunostimulatory nucleotide sequence contains a CpG motif.

12. The method of claim 11 wherein the immunostimulatory nucleotide is selected from the group consisting of CpG 2006 (SEQ ID NO: 1), CpG 2216 (SEQ ID NO: 2), AAC-30 (SEQ ID NO: 3), and GAC-30 (SEQ. ID NO.: 4).

13. The method of claim 10 wherein the immunostimulatory nucleotide sequence is stabilized by structure modification such as phosphorothioate-modification.

14. The method of claim 10 wherein the immunostimulatory nucleotide sequence is encapsulated in cationic liposomes.

15. The method of claim 1 wherein the tumor-derived DC inhibitory factor antagonist is an anti-IL-10R monoclonal antibody and the TLR agonist is CpG 2006 (SEQ ID NO: 1).

16. The method of claim 1, further comprising administering a substance which allows for slow release of the tumor-derived DC inhibitory factor antagonist and/or TLR agonist at a delivery site.

17. The method of claim 1, wherein the tumor-derived DC inhibitory factor antagonist and/or TLR agonist is administered intravenously, intratumorally, intradermally, intramuscularly, subcutaneously, or topically.

18. The method of claim 1 further comprising administering at least one tumor-associated antigen.

19. The method of claim 18 wherein the tumor-associated antigen is linked to the TLR agonist.

20. The method of claim 18 wherein the tumor-associated antigen is selected from the group consisting of Melan-A, tyrosinase, p97, β-HCG, GalNAc, MAGE-1, MAGE-2, MAGE-3, MAGE-4, MAGE-12, MART-1, MUC1, MUC2, MUC3, MUC4, MUC18, CEA, DDC, melanoma antigen gp75, HKer 8, high molecular weight melanoma antigen, K19, Tyr1 and Tyr2, members of the pMel 17 gene family, c-Met, PSA, PSM, α-fetoprotein, thyroperoxidase, gp100, NY-ESO-1, p53 and telomerase.

21. The method of claim 1 wherein the cancer to be treated is selected from the group consisting of melanoma, breast, pancreatic, colon, lung, glioma, hepatocellular, endometrial, gastric, intestinal, renal, prostate, thyroid, ovarian, testicular, liver, head and neck, colorectal, esophagus, stomach, eye, bladder, glioblastoma, and metastatic carcinomas.

22. The method of claim 1 further comprising administering an activating agent.

23. The method of claim 22 wherein the activating agent is selected from the group consisting of IFNα, TNFα, RANK ligand/agonist, CD40 ligand/agonist or a ligand/agonist of another member of the TNF/CD40 receptor family.

24. The method of claim 1 further comprising administering a cytokine which increases the number of blood dendritic cells.

25. The method of claim 24 wherein the dendritic cell proliferation agent is selected from the group consisting of FLT3-L, GM-CSF and G-CSF.

26. The method of claim 1 further comprising delivering to the tumor a chemokine active on dendritic cells.

27. The method of claim 26 wherein the chemokine is selected from the group consisting of: CCL21, CCL3, CCL20, CCL16, CCL5, CCL25, CXCL12, CCL7, CCL8, CCL2, CCL13, CXCL9, CXCL10 and CXCL11.

28. The method of claim 26 wherein the chemokine is delivered to the tumor using a targeting construct comprising a chemokine or a biologically active fragment or variant thereof and a targeting moiety.

29. The method of claim 28 wherein the targeting moiety is selected from the group consisting of:
a) a peptide of at least 10 amino acids;
b) a protein;
c) a small molecule;
d) a vector; and
e) an antibody or antibody fragment.

30. The method of claim 1 wherein the tumor-derived DC inhibitory factor antagonist and/or the TLR agonist are linked to each other.

31. The method of claim 30, wherein the tumor-derived DC inhibitory factor antagonist and/or the TLR agonist are further linked to a tumor associated antigen.


SEQUENCE LISTING (5 sequences)

SEQ ID NO: 1 - DNA, 23 bp, Artificial Sequence
  oligonucleotide
tcgtcgtttgtcgttttgtcgtt

SEQ ID NO: 2 - DNA, 20 bp, Artificial Sequence
  oligonucleotide
gggggacgatcgtcgggggg

SEQ ID NO: 3 - DNA, 30 bp, Artificial Sequence
  oligonucleotide
accgataacgttgccggtgacggcaccacg

SEQ ID NO: 4 - DNA, 30 bp, Artificial Sequence
  oligonucleotide
accgatgacgtcgccggtgacggcaccacg

SEQ ID NO: 5 - DNA, 20 bp, Artificial Sequence
  oligonucleotide
tccatgacgttcctgatgct